SEQUENCE LISTING (6 sequences)

SEQ ID NO: 1 - protein, 260 aa, Artificial Sequence
  DsRed-Express-Streptag I fusion protein
MTMITPSLHACRSTLEDPRVPVATMASSEDVIKEFMRFKVRMEGSVNGHEFEIEGEGEGRPYEGTQTAKLKVTKGGPLPFAWDILSPQFQYGSKVYVKHPADIPDYKKLSFPEGFKWERVMNFEDGGVVTVTQDSSLQDGSFIYKVKFIGVNFPSDGPVMQKKTMGWEASTERLYPRDGVLKGEIHKALKLKDGGHYLVEFKSIYMAKKPVQLPGYYYVDSKLDITSHNEDYTIVEQYERAEGRHHLFLRSAWRHPQFGG

SEQ ID NO: 2 - protein, 339 aa, Artificial Sequence
  ALAD-Streptag I fusion protein
  VARIANT 164: Xaa = Any Amino Acid
MTMGSMTDLIQRPRRLRKSPALRAMFEETTLSLNDLVLPIFVEEEIDDYKAVEAPGVMRIPEKHLAREIERIANAGIRSVMTFGISHHTDETGSDAWREDGLVARMSRICKQTVPEMIVMSDTCFCEYTSHGHCGVLCEHGVDNDATLENLGKQAVVAAAAGAXFIAPSAAMDGQVQAIRQALDAAGFKDTAIMSYSTKFASSFYGPFREAAGSALKGDRKSYQMNPMNRREAIRESLLDEAQGANCLMVKPAGAYLDIVRELRERTELPIGAYQVSGEYAMIKFAALAGAIDEEKVVLESLGSIKRAGADLIFSYFALDLAEKKILRRSAWRHPQFGG

SEQ ID NO: 3 - DNA, 32 bp, Artificial Sequence
  Primer for amplification of the ferritin gene
ccttagtcgaattcatgacgaccgcgtccacc

SEQ ID NO: 4 - DNA, 33 bp, Artificial Sequence
  Primer for amplification of the ferritin gene
gggaaattagccctcgagttagctttcattatc

SEQ ID NO: 5 - DNA, 33 bp, Artificial Sequence
  Primer for amplification of the pure gene
gttttaagacccatggcttcccgcaataatccg

SEQ ID NO: 6 - DNA, 30 bp, Artificial Sequence
  Primer for amplification of the pure gene
cgcaaacctggatcctgccgcacctcgcgg

(19) United States
(12) Patent Application Publication
(10) Pub. No.: US 2008/0097080 A1
(43) Pub. Date: Apr. 24, 2008

(54) Protein structure

(76) Inventors: John Charles Sinclair, Oxford (GB); Martin Edward Mantyla Noble, Oxford (GB)

Correspondence Address: HAMILTON, BROOK, SMITH & REYNOLDS, P.C., 530 VIRGINIA ROAD, P.O. BOX 9133, CONCORD, MA, 01742-9133, US

(21) Appl. No.: 11/807,922

(22) Filed: May 30, 2007

Related U.S. Application Data
(63) Continuation-in-part of application No. 10/530,795, filed on Nov. 7, 2005.

(30) Foreign Application Priority Data
Oct. 8, 2002 (GB) 022323.7

(51) Int. Cl. C07K 14/00; C12N 1/00; C12N 15/62; C12N 15/63; G06F 19/00

(52) U.S. Cl. 530/350; 435/243; 435/320.1; 536/23.4; 702/19

(57) ABSTRACT

Protein structures 1 repeating regularly in one, two or three dimensions comprise protein protomers 2 which each comprise at least two monomers 5, 6 genetically fused together. The monomers 5, 6 are monomers of respective oligomer assemblies 3, 4 into which the monomers are assembled to assembly of the protein structure. The first oligomer assembly 3 has rotational symmetry axes including a set of rotational symmetry axes of order N, where N equals 2, 3, 4 or 6. The second oligomer assembly 4 has a rotational symmetry axis of the same order N as said set of rotational symmetry axes of said first oligomer assembly 3. Due to the symmetry of the oligomer assemblies 3, 4, the rotational symmetry axis axes of each second oligomer assembly 4 is aligned with one of said set of rotational symmetry axes of a first oligomer assembly 3 with N protomers being arranged symmetrically therearound. Thus, an N-fold fusion between the oligomer assemblies 3, 4 is produced and the arrangements of the rotational symmetry axes of the oligomer assemblies 3, 4 cause the protein structure to repeat regularly. The protein structure has many uses, for example to support molecular entities for x-ray crystallography or electron microscopy.

### CROSS-REFERENCE TO RELATED APPLICATIONS

This application is a Continuation-In-Part of co-pending application Ser. No. 10/530,795, which is itself the US national phase of International Patent Application No. PCT/GB03/04306, filed Oct. 8, 2003.

### REFERENCE TO SEQUENCE LISTINGS

SEQ ID NO. 1 is DsRed-Express-Streptag I fusion protein used in the examples.

SEQ ID NO. 2 is ALAD-Streptag I fusion protein used in the examples.

SEQ ID NO. 3 is a primer for amplification of the ferritin gene used in the examples.

SEQ ID NO. 4 is a further primer for amplification of the ferritin gene used in the examples.

SEQ ID NO. 5 is a primer for amplification of the PurE gene used in the examples.

SEQ ID NO. 6 is a primer for amplification of the PurE gene used in the examples.

### BACKGROUND OF THE INVENTION

1. Field of the Invention

The present invention relates to protein structures which repeat regularly in one, two or three dimensions. The protein structures are based on symmetrical oligomer assemblies capable of self-assembly from the monomers of the oligomer assembly. Such protein structures may be lattices which repeat in three dimensions, layers which repeat in two dimensions or chains which repeat in one dimension. The layers or lattices may have pores with dimensions of the order of nanometres to hundreds of nanometres. The protein structures are nanostructures which have many potential uses, for example as a matrix to support molecular entities for X-ray crystallography.

2. Description of Related Art

WO-00/68248 discloses regular protein structures based on symmetrical oligomer assemblies capable of self-assembly. In particular, WO-00/68248 discloses structures formed from protein protomers (referred to as a “fusion protein” in WO-00/68248) comprising at least two monomers (referred to as “oligomerization domains” in WO-00/68248) which are each monomers of a respective symmetrical oligomer assembly. Self-assembly of the monomers into the oligomer assembly causes assembly of the regular structures themselves. Several different types of structures are disclosed, including discrete structures and structures extending in one, two and three dimensions.

In WO-00/68248, the relative orientations of the monomers within the protomers are selected to provide the desired regular structure upon self-assembly. The monomers are fused together through a rigid linking group which is carefully selected to provide the requisite relative orientation of the monomers in the protomer. For example, in the laboratory production reported in WO-00/68248, the selection of the protomer was performed using a computer program to model monomers connected by a linking group in the form of a continuous, intervening alpha-helical segment over a range of incrementally increased lengths. Thus, for example, the lattices suggested in WO-00/68248 having a regular structure repeating in three dimensions are formed from protomers comprising two monomers of respective dimeric or trimeric oligomer assemblies which are symmetrical about a single rotational axis. The relative orientation of the two monomers is selected to provide a specific angle of intersection between the rotational symmetry axis of the two oligomer assemblies. Thus, there is a single fusion between the two oligomer assemblies and the relative orientation of the oligomer assemblies is controlled by careful selection of the linking group providing the fusion.

WO-00/68248 only reports laboratory production of protein structures of a discrete cage and a filament extending in one dimension. It is expected that application of the teaching of WO-00/68248 to protein lattices repeating in three dimensions would encounter the following difficulties. Firstly, it is expected that there would be a difficulty in design arising from the requirement to select the relative orientation of the monomers within the protomer appropriate for constructing a lattice. This would probably reduce the numbers of types of oligomer assembly available to form a protein lattice, and hence make it difficult to identify suitable proteins. Secondly, it is expected that practical difficulties would be encountered during assembly. The structures disclosed in WO-00/68248 rely on the rigidity of the fusion between monomers in protomers which forms the single fusion between oligomer assemblies. WO-00/68248 teaches that the relative orientation of the monomers in the protomers controls the relative orientation of the oligomer assemblies in the resultant structure, so it is expected that flexing of the fusion away from the desired relative orientation would reduce the reliability of self-assembly. It is expected that such a problem would become more acute as the size of the repeating unit increases, thereby providing a practical restriction on the reliable production of lattices with a relatively large pore sizes. Similar problems also restrict the design and manufacture of one and two dimensional structures.

Accordingly, it would be desirable to provide protein structures having a different type of structure in which these expected problems might be alleviated.

### BRIEF SUMMARY OF THE INVENTION

According to an aspect of a present invention, there is provided a protein structure which repeats regularly in one, two or three dimensions,

the protein structure comprising protein protomers which each comprise at least two monomers genetically fused together, the monomers each being monomers of a respective oligomer assembly, the protomers comprising:

a first monomer which is a monomer of a first oligomer assembly having rotational symmetry axes extending in at least two dimensions, including a set of rotational symmetry axes of order N, where N equals 2, 3, 4 or 6; and

a second monomer genetically fused to said first monomer which second monomer is a monomer of a second oligomer assembly having a rotational symmetry axis of the same order N as said set of rotational symmetry axes of said first oligomer assembly,

the first monomers of the protomers are assembled into said first oligomer assemblies and the second monomers of the protomers are assembled into said second oligomer assemblies, said rotational symmetry axis of said second oligomer assemblies of order N being aligned with one of said set of rotational symmetry axes of order N of one of said first oligomer assemblies with N protomers being arranged symmetrically therearound, the arrangements of the rotational symmetry axes of the first oligomer assembly and the second oligomer assembly causing the protein structure to repeat regularly in one, two or three dimensions.

As a result of using a second oligomer assembly having a rotational symmetry axis of the same order N as said set of rotational symmetry axes of said first oligomer assembly, the oligomer assemblies are fused with those symmetry axes being aligned and with N protomers arranged symmetrically therearound. This means that there is an N-fold fusion between the first and second oligomer assemblies. Furthermore the repeating pattern of the protein structure is derived from arrangements of the rotational symmetry axes of the first oligomer assembly and the second oligomer assembly. In particular, it is not dependent on the relative orientation of the monomers within the protomer.

Therefore, protein structures in accordance with the present invention may be designed by selecting oligomers assemblies with appropriate symmetry to build a structure repeating in one, two or three dimensions, as desired. Depending on the symmetries of the oligomer assemblies chose, the structures may be lattices which repeat in three dimensions, layers which repeat in two dimensions or chains which repeat in one dimension.

Protomers are then produced comprising monomers of the selected oligomer assemblies fused together. Subsequently, the protomers are allowed to self-assemble under suitable conditions.

To assist in understanding, reference is made to FIG. 1 which illustrates a particular example of a protein structure which is a lattice 1 in accordance with the present invention, as described in more detail below. In particular, the protein lattice 1 has a comprises a first oligomer assembly 3 which has a set of rotational symmetry axes of order 4 (amongst others), which in this example is human heavy chain ferritin which has octahedral symmetry, so having a set of rotational symmetry axes of order 4 (amongst others). Each of the monomers 5 of the first oligomer assembly 3 is fused to a second monomer 6 of a second oligomer assembly 4, which in this example is E. Coli PurE which has symmetry belonging to the dihedral D₄ point group 4, so having a rotational symmetry axis of order 4. As a result, the second monomers 6 are assembled into the second oligomer assemblies 4 arranged with their rotational symmetry axes of order 4 aligned along the rotational symmetry axes of order 4 of the first oligomer assembly 3, and with a 4-fold fusion between the first and second oligomer assemblies 3 and 4. Thus, the symmetry of the protein lattice 1 is the same as the symmetry of the set of rotational symmetry axes of order 4, as will be described in more detail below.

Accordingly, the present invention involves the use of a different class of oligomers assemblies from that used in WO-00/68248. The present invention provides the benefit that one is not restricted by the need to control the relative orientation of the monomers within the protomer. Thus the design of protein structure is assisted in that the relative orientation of the monomers withing the protomer is a less critical constraint. Similarly, more reliable assembly of the protein structure is possible, as described in more detail below.

According to other aspects of the present invention, there is provided an individual protomer or plural protomers capable of self-assembly to form such a protein structure, as well as polynucleotides encoding such protomers, vectors and host cells capable of expressing such promoters and methods of making the protomers.

The present invention will now be described in more detail by way of non-limitative example with reference to the accompanying drawings.

### BRIEF DESCRIPTION OF THE DRAWINGS

FIG. 1 is a diagram schematically illustrating, for a first protein lattice, the design of a homologous protomer based on two oligomer assemblies and production of the lattice itself;

FIG. 2 is a diagram schematically illustrating, for a second protein lattice, the design of two heterologous protomers based on three oligomer assemblies and production of the lattice itself;

FIG. 3 is a picture of an experimentally produced protein lattice of the type illustrated in FIG. 1;

FIG. 4 is an electron micrograph of a specific protein chain which has been prepared;

FIG. 5 is an electron micrograph of a specific protein layer which has been prepared;

FIG. 6 is an electron micrograph which is an enlargement of an area of FIG. 5; and

FIG. 7 is an electron micrograph of FIG. 6 after an image enhancement procedure.

### DETAILED DESCRIPTION OF THE INVENTION

Protein structures in accordance with the present invention may be designed by selecting oligomer assemblies which, when fused together with rotational symmetry axes of the same order aligned with each other, produce a repeating unit which is capable of repeating in one, two or three dimensions. As the symmetry of the repeating unit, and hence the structure as a whole, depends on the symmetry of the oligomer assemblies, this involves a selection of oligomer assemblies having a quaternary structure which provides appropriate symmetries. This is a straightforward task, because the symmetries of oligomer assemblies are generally available in the scientific literature on proteins, for example from The Protein Data Bank; H. M. Berman, J. Westbrook, Z. Feng, G. Gilliland, T. N. Bhat, H. Weissig, I. N. Shindyalov & P. E. Bourne; Nucleic Acids Research, 28 pp. 235-242 (2000) which is the single worldwide archive of structure data of biological macromolecules, also available through websites such as http://www.rcsb.org.

In some cases, the repeating unit repeats in the same orientation across the structure. In other cases, two or more adjacent repeating units together form a unit cell which repeats in the same orientation across the structure, but with the repeating units within a unit cell arranged in different orientations.

Examples of oligomer assemblies which produce structures which repeat regularly in three dimension are given below.

The first oligomer assembly has a quaternary structure with rotational symmetry axes extending in at least two dimensions, including a set of rotational symmetry axes of order N, where N equals 2, 3, 4 or 6. The second oligomer assembly has a quaternary structure with a rotational symmetry axis of the same order N as said set of rotational symmetry axes of said first oligomer assembly.

In the assembled first oligomer assembly, inevitably and by definition, there are groups of first monomers arranged symmetrically around each of the set of rotational symmetry axes of order N of the first oligomer assembly. This is because the symmetry results from the identical monomers being so arranged around the rotational symmetry axes.

As a result of the second monomers fused to the first oligomer assembly being arranged symmetrically around one of the set of rotational symmetry axes of order N of the first oligomer assembly, it follows that the second oligomer assembly is held with the group of fused second monomers also held symmetrically around that one of the set of rotational symmetry axes of the first oligomer assembly.

However, inevitably and by definition, the second monomers also assemble in the second oligomer assembly in a symmetrical arrangement around the rotational symmetry axis of order N of the second oligomer assembly. Thus, the result of the second oligomer assembly having a rotational symmetry axis of the same order N as the set of rotational symmetry axes of the first oligomer assembly is that the first and second oligomer assemblies assemble with their symmetry axes aligned with one another. It follows from the symmetry of both oligomer assemblies that this is the most stable arrangement. This results in an N-fold fusion between the first and second oligomer assemblies, where N is a plural number equal to the order of the respective rotational symmetry axis of the first oligomer assembly and the rotational symmetry axis of the second oligomer assembly. In each of the first and second oligomer assemblies, there are N monomers arranged around the rotational symmetry axis, each of the monomers being fused within a respective protomer to a monomer of the other oligomer assembly.

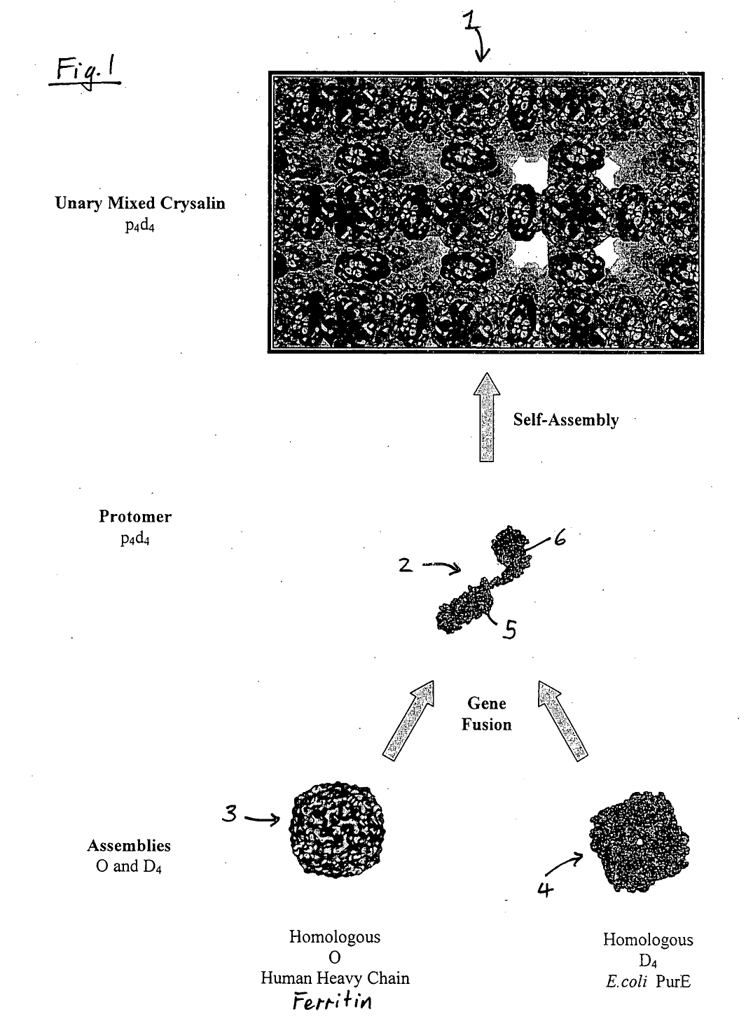

Thus the set of rotational symmetry axes does not include all the rotational symmetry axes of the first oligomer assembly. Rather the set comprises the rotational symmetry axes of the first oligomer assembly which are of the same order as rotational symmetry axes of the second oligomer assembly. For example in the example of FIG. 1, the set of rotational symmetry axes of the first oligomer assembly 3 are the rotational symmetry axes of order 4, rather than those of order 3 or 2, due to the second oligomer assembly 4 having rotational symmetry axes of order 4. Further examples are given below.

The particular choice of symmetries of the first and second oligomer assemblies results, on assembly of the protomers into the structure, in the oligomer assemblies being built up with their rotational symmetry axes aligned. Thus, the relative arrangement of the fused oligomer assemblies and hence the protein structure as a whole are therefore derived from arrangements of the rotational symmetry axes of the first oligomer assembly and the second oligomer assembly. In particular, it is not dependent on the relative orientation of the monomers within the protomer. In other words, the present invention provides the advantage that the one, two or three dimensional repeating pattern of the protein structure may be based solely on the arrangements of the rotational symmetry axes of the oligomer assemblies. This provides advantages in the design of the protein structures by making it easy to select appropriate oligomer assemblies for use in the protein structure. During design, the relative orientation of the monomers within an individual protomer in its unassembled form becomes a much lower constraint than is present in, for example, WO-00/68248.

There are also advantages during self-assembly of the structure. In particular, the formation of an N-fold fusion between two given oligomer assemblies results in the bond between the two oligomer assemblies being relatively rigid. This reduces relative motion of the oligomer assemblies during the assembly process and assists in reliable formation of the structure with the oligomer assemblies in the correct relative positions.

Although there are particular advantages in the use of a second oligomer assembly which has a rotational symmetry axis of the same order as the rotational symmetry axes of the first oligomer assembly, this is not essential. Alternatively, it would be possible for the second monomers arranged symmetrically around the rotational symmetry axes of the first oligomer assembly to be monomers of separate oligomer assemblies, for example of dimeric oligomer assemblies (being heterologous or homologous). In that case, the second oligomer assembly would effectively be replaced by a group of separate dimeric oligomer assemblies, equal in number to the order of the rotational symmetry axis of the first oligomer assembly, with the separate dimeric oligomer assemblies held around the rotational symmetry axis of the first oligomer assembly in an arrangement which might or might not have the N-fold symmetry of the rotational symmetry axis of the first oligomer assembly.

The form and production of the protomers will now be described. Except that the present invention involves protomers in which are different in that they comprise different monomers from WO-00/68248, the form and production of the protomers per se, as well as the polynucleotide encoding the protomers, may be as the same as disclosed in WO-00/68248 which is therefore incorporated herein by the reference.

The nature of the monomers themselves will now be described.

The monomers are monomers of oligomer assemblies which are capable of self-assembly under suitable conditions to produce a protein structure. The secondary and tertiary structure of the monomers is unimportant in itself providing they assemble into a quaternary structure with the required symmetry. However, it is advantageous if the protein is easily expressed and folded in an heterologous expression system (for example using plasmid expression vector in E. Coli).

The monomers may be naturally occurring proteins, or may be modified by peptide elements being absent from, substituted in, or added to a naturally occurring protein provided that the modifications do not substantially affect the assembly of the monomers into their respective oligomer assembly. Such modifications are in themselves known for a number of different purposes which may be applied to monomers of the present invention. In other words, the monomer may be a homologue and/or fragment and/or fusion protein of a naturally occurring protein.

The monomer may be chemically modified, e.g. post-translationally modified. For example, it may be glycosylated or comprise modified amino acid residues.

Although the monomers may be fused directly together, preferably the monomers are fused by a linking group of peptide or non-peptide elements. In general, linking two proteins by a linking group is known for other purposes and such linking groups may be applied to the present invention.

Another factor in the selection of appropriate oligomer assemblies is the location and orientation of (a) the termini of the first monomers when arranged in the first oligomer assembly in its natural form (i.e. not fused to a second oligomer assembly) and (b) the termini of the second monomers when arranged in the second oligomer assembly in its natural form (i.e. not fused to the first oligomer assembly). Such information on the arrangement of the termini in the oligomer assembly in its natural form is generally available for oligomer assemblies, for example from The Protein Data Bank referred to above. Ideally, these termini should have the same separation and orientation, because they will be fused together in the assembled protein structure to constitute the N-fold fusion arranged symmetrically around a rotational symmetry axis. That being said, it is not essential for the separation and orientation to be the same, because any difference may be accommodated by deformation of the monomers near the N-fold fusion and/or by use of a linking group. Therefore, as a general point, oligomer assemblies should be chosen in which the termini of both oligomer assemblies which are to be fused together in an N-fold fusion allows formation of the fusion without preventing assembly of the oligomer assemblies and hence the protein structure.

Considering the deformation of the monomers near the N-fold fusion mentioned above, it is desirable to minimise such deformation which will tend to reduce the reliability of the assembly process. However, if a linking group is fused between the monomers, such deformation may be taken up, at least partially, by the linking group itself. This reduces the deformation of the monomers, thereby increasing the reliability of self-assembly because the linking group does not take part in the assembly process as regards to not being part of the naturally occurring protein. There is a particular advantage of the use of a linking group.

Furthermore, the linking group may be specifically designed to be oriented relative to the first and second monomers in the protomer in its normal form, prior to assembly, to reduce such differences in the position and/or orientation of the termini of the first and second monomers. Using position and orientation of the termini of the first and second monomers in the first and second oligomer assemblies in their natural form which is generally available for oligomer assemblies, as discussed above, it is possible to design an appropriate linking group using conventional modelling techniques.

Typically, the monomers are fused at their end termini. Alternatively, the monomers may be fused at an alternative location in the polypeptide chain so long as the native fold and symmetry of the naturally occurring oligomer assembly remains the same. For example, one of the monomers may be inserted into a structurally tolerant portion of the other monomer, for example in a loop extending out of the oligomer assembly. Also, truncation of a monomer is feasible and may be estimated by structural examination.

Some examples of symmetries for the oligomer assemblies to produce a protein structure are as follows.

First there will be described examples to produce a protein lattice which repeats in three dimensions. In the case of a protein lattice, the first oligomer assembly has rotational symmetry axes which extend in three dimensions.

In these examples, the first oligomer assembly belongs to one of a tetrahedral point group, an octahedral point group or a dihedral point group of order O, where O equals 3, 4 or 6.

In some classes of protein lattice, the protomers are homologous with respect to the monomers, ie there is a single type of protomer within the protein lattice. For example, Table 1 represents some simple homologous protomers capable of forming a protein lattice.

TABLE 1
Homologous Protomers

| Protomer | Class Name | M | N |
|---|---|---|---|
| p₃p₃ | Platonic | 12 | 3 |
| p₄p₄ | Platonic | 24 | 4 |
| p₄p₃ | Platonic | 24 (or 12) | 3 |
| p₃d₃ | Mixed | 12 | 3 |
| p₃d₂ | Mixed | 12 | 2 |
| p₄d₄ | Mixed | 24 | 4 |
| p₄d₃ | Mixed | 24 | 3 |
| p₄d₂ | Mixed | 24 | 2 |
| d₃d₃d₂ | Dihedral | 6 | 3, 2 |
| d₄d₄d₂ | Dihedral | 8 | 4, 2 |
| d₆d₆d₂ | Dihedral | 12 | 6, 2 |

In Table 1, each protomer is identified by letters which represent the respective monomers of the protomer. In particular the letters identify the point group to which the oligomer assembly of that monomer belongs. For each letter, the subscript number represents the order of the point group. The letter p represents a platonic point group, so p₃ represents a tetrahedral point group, and p₄ represents an octahedral point group. The letter d represents a dihedral point group.

In the final two columns of the table, there is given the number M of first monomers in the first oligomer assembly and the order(s) N of the set of rotational symmetry axes of the first oligomer assembly. N is also the order of the rotational symmetry axis of the second oligomer assembly aligned with a respective rotational symmetry axis of the first oligomer assembly, and around which there is formed an N-fold fusion between the first and second oligomer assemblies.

The protomers have been divided into classes which have been named according to the nature of the monomers of the proteins for ease of reference.

In both the platonic and mixed classes, the first oligomer assembly belongs to a platonic point group, which is either a tetrahedral point group or an octahedral point group.

In the mixed class, the second monomer is a monomer of an oligomer assembly belonging to a dihedral point group. In each case, the order N of the dihedral point group, which is the order of the principal rotational symmetry axis of the dihedral point group, is equal to the order of one of the rotational symmetry axes of the first oligomer assembly. This may either be the principal rotational symmetry axis of the first oligomer assembly or one of the rotational symmetry axes of the first oligomer assembly of lower order. The rotational symmetry axes of the first oligomer assembly of order N therefore constitute the set of rotational symmetry axes of the first oligomer assembly. The symmetries of the first and second oligomer assemblies results in the formation of a unit cell in which the principal rotational symmetry axis of each second oligomer assembly belonging to a dihedral point group is aligned with one of set of rotational symmetry axes of order N of the platonic point group, with an N-fold fusion therebetween, in the manner described above.

The protein lattices of the mixed class are the easiest to visualise. In particular, the first oligomer assembly belonging to a platonic point group may be visualised as a node from which the set of rotational symmetry axes of order N extend outwardly. The dihedral point groups may be visualised as linear links with the principal rotational symmetry axis of the dihedral point group aligned with one of the set of rotational symmetry axes of order N of the first oligomer assembly. In this way, it is easy to visualise the formation of the lattice with pores in the spaces between the oligomer assemblies.

FIG. 1 illustrates a particular example of a protein lattice 1 belonging to the mixed class, in particular having a protomer 2 represented by p₄d₄. The first oligomer assembly 3 is human ferritin heavy chain (HFH) which belongs to an octahedral point group, so having a set of rotational symmetry axes of order 4 (amongst others). The second oligomer assembly is E. Coli PurE which belongs to a dihedral D₄ point group of order 4, so having a rotational symmetry axis of order 4. The protomer comprises a first monomer 5 of the first oligomer assembly 3 and a second monomer 6 of the second oligomer assembly 4 fused together. On assembly, the protomers 2 form a lattice 1 which repeats regularly in three dimensions. The repeating unit (which is also a unit cell) may be taken as, for example, one of the first oligomer assemblies 3, together with and half of each of the adjacent second oligomer assemblies 4 formed by the second monomers 6 fused to the first monomers 5 of that first oligomer assembly 1. As clearly visible from FIG. 1, the symmetry of the protein lattice 1 is based on the arrangement of the rotational symmetry axes of order 4 of the first oligomer assembly 3 and the second oligomer assembly 4. This is because the rotational symmetry axes of order 4 of the second oligomer assembly 4 are aligned with the set of rotational symmetry axes of order 4 of the first oligomer assembly 3. The symmetry of the lattice 1 is the same as the symmetry of the set of rotational symmetry axes of order 4 of the first oligomer assembly 4.

In the platonic class, the second oligomer assembly belongs to a platonic point group as well as the first oligomer assembly.

In the first two protein lattices where the protomers belong to platonic point groups of the same order, the first and second oligomer assemblies may be identical, in which case the first and second monomers are also identical, or may be different oligomer assemblies belonging to an identical point group. The set of rotational symmetry axes of order N around which is formed an N-fold fusion are the principal rotational symmetry axes of the two oligomer assemblies.

In the third protein lattice in the platonic class where the first and second oligomer assemblies belong respectively to tetrahedral and octahedral point groups (or vice versa), the rotational symmetry axes of order N around which the N-fold fusion occurs are the rotational symmetry axes of order 3 of the two oligomer assemblies. In this case, either one of the oligomer assemblies may be considered as the first oligomer assembly. If the oligomer assembly belonging to a tetrahedral point group is considered as the first oligomer assembly, then the set of rotational symmetry axes are the principal rotational symmetry axes. If the oligomer assembly belonging to an octahedral point group is considered as the first oligomer assembly, then the set of rotational symmetry axes are the set of rotational symmetry axes of order 3, because this is the order of the rotational symmetry axes of the second oligomer assembly belonging to the tetrahedral point group.

The platonic class may be visualised by considering each oligomer assembly as a node from which the set of rotational symmetry axes of order N extend outwardly and joined to the rotational symmetry axes of an oligomer assembly of the opposite type.

Lastly, in the dihedral class, the protomers' comprise three monomers all belonging to a dihedral point group. The central monomer is fused at each terminus to the other two monomers.

The central monomer may be considered as the first monomer of a first oligomer assembly belonging to a dihedral point group of order O, where O equals 3, 4 or 6.

The left hand monomer may considered as the second monomer being a monomer of a second oligomer assembly belonging to a dihedral point group of the same order O as the dihedral point group of the first oligomer assembly. Thus, as a result of the symmetries of the first oligomer assembly and this one of the second oligomer assembly, this results of the formation of a repeating unit in which the principal rotational symmetry axes of both oligomer assemblies (i.e. the rotational symmetry axis of the same order as the dihedral point group) are aligned. Thus in this example N equals O. Therefore, in the protein lattice, these oligomer assemblies are arranged in columns along which the first and second oligomer assemblies are alternately arranged.

The right hand monomer may considered as a third monomer being a monomer of an oligomer assembly belonging to a dihedral point group of order 2 and so have a rotational symmetry axis of order 2 which is equal to the rotational symmetry axis of order 2 of the first oligomer assembly. Such rotational symmetry axes of the first oligomer assembly are equal in number to the order of the dihedral point group to which the first oligomer assembly belongs, and extend perpendicular to the principal rotational symmetry axis of the dihedral point group, being arranged symmetrically around that principal rotational symmetry axis. Therefore, the second oligomer assemblies belonging to a dihedral point group of order 2 are arranged in the assembled protein lattice with their principal rotational symmetry axes aligned to the just described rotational symmetry axes of order 2 of the first oligomer assembly. As these extend perpendicular to the principal rotational symmetry axes of the first oligomer assembly, the second oligomer assemblies belonging to a dihedral point group of order 2 may be considered as links between the columns of oligomer assemblies described above.

In other classes of protein lattice, the protomers are heterologous with respect to the monomers i.e. there are two or more types of protomer in the protein lattice. To achieve assembly of any two types of protomer, the two types of protomer include different monomers of the same heterologous oligomer assembly. Thus when the protomers of the different types are allowed to assemble, the heterologous oligomer assemblies assemble, thereby linking the protomers of the two types. However, in contrast to homologous protomers, a single type of protomer cannot by itself assemble into the entire protein lattice. The individual monomers of the heterologous oligomer assembly cannot self-assemble into the entire heterologous oligomer assembly in the absence of the other, different monomers of that heterologous assembly. This provides advantages during manufacture of the protein lattices, because each type of protomer may be separately produced without assembly of an entire protein lattice which might otherwise disrupt the production of the protomer. This allows production in a two-stage process, which will be described in more detail below.

Preferably, the heterologous oligomer assembly belongs to a cyclic point group. In this case, the heterologous oligomer assembly may constitute the second oligomer assembly which is fused in the assembled lattice by an N-fold fusion to the first oligomer assembly.

In the simplest types of protein lattice, the heterologous protomers of each type further comprise a monomer of a homologous oligomer assembly, which may be the first oligomer assembly of that type of protomer. The individual types of protomer may assemble into a respective, discrete component of the unit cell, as a result of the monomers of the homologous oligomer assembly self-assembling. This is an advantage of the heterologous protomers, because assembly of the lattice may be avoided until the components are brought together. Otherwise assembly of the lattice might hinder the production of the protomers themselves.

For example, Table 2 represents some simple heterologous protomers capable of forming a protein lattice.

TABLE 2
Heterologous Protomers

| | | | 1st | | 2nd | |
|---|---|---|---|---|---|---|
| | | | Protomer | | Protomer | |
| Protomer | Components | Name | M | N | M | N |
| p₃c_{3A} + p₃c_{3A*} | P₃/P₃ | Platonic | 12 | 3 | 12 | 3 |
| p₄c_{3A} + p₃c_{3A*} | P₄/P₄ | Platonic | 24 | 3 | 12 | 3 |
| p₄c_{3A} + p3_{c3A*} | P₄/P₃ | Platonic | 24 | 3 | 12 | 3 |
| p₃c_{3A} + d₃c_{3A*} | P₃/D₃ | Mixed | 12 | 3 | | |
| p₃c_{2A} + d₂c_{2A*} | P₃/D₂ | Mixed | 12 | 2 | | |
| p₄c_{4A} + d₄c_{4A*} | P₄/D₄ | Mixed | 24 | 4 | | |
| p₄c_{3A} + d₃c_{3A*} | P₄/D₃ | Mixed | 24 | 3 | | |
| p₄c_{2A} + d₂c_{2A*} | P₄/D₂ | Mixed | 24 | 2 | | |
| c_{3A}d₃d₂ + c_{3A*}d₃d₂ | D₃/D₃ | Dihedral | 6 | 3, 2 | 6 | 3, 2 |
| c_{4A}d₄d₂ + c_{4A*}d₄d₂ | D₃/D₃ | Dihedral | 8 | 4, 2 | 8 | 4, 2 |
| c_{6A}d₆d₂ + c_{6A*}d₆d₂ | D₆/D₆ | Dihedral | 12 | 6, 2 | 12 | 6, 2 |
| d₃d₃c_{2A} + d₃d₃ c_{2A*} | D₃/D₃ | Dihedral | 6 | 3, 2 | 6 | 3, 2 |
| d₄d₄c_{2A} + d₄d₄ c_{2A*} | D₄/D₄ | Dihedral | 8 | 4, 2 | 8 | 4, 2 |
| d₆d₆c_{2A} + d₆d₆ c_{2A*} | D₆/D₆ | Dihedral | 12 | 6, 2 | 12 | 6, 2 |
| c_{3A}d₃c_{2B} + c_{3A*} d₃c_{2B*} | D₃/D₃ | Dihedral | 6 | 3, 2 | 6 | 3, 2 |
| c_{4A}d₄c_{2B} + c_{4A*} d₄c_{2B*} | D₄/D₄ | Dihedral | 8 | 4, 2 | 8 | 4, 2 |
| c_{6A}d₆c_{2B} + c_{6A*} d₆c_{2B*} | D₆/D₆ | Dihedral | 12 | 6, 2 | 12 | 6, 2 |

In Table 2, monomers of a single heterologous oligomer assembly belonging to a cyclic point group are used so that the protein lattice is formed from two types of protomer identified in the first column. Each of the protomers includes one of the monomers of the heterologous oligomer assembly.

In Table 2, the monomers of each protomer are identified by lower case letters in similar manner as in Table 1. The lower case letters p and d have the same meaning as in Table 1. In addition, lower case c represents a monomer of a heterologous oligomer assembly belonging to a cyclic point group. The subscript number again represents the order of the point group. The subscript capital letters A and A*are used to identify the two different monomers of the same heterologous assembly.

In Table 2, the second column identifies the point groups to which the components resulting from the assembly of each type of protomer belongs. A similar notation is used as for the monomers of the protomer, except that capital letters are used to indicate that the point group of the component is being referred to. Thus capital letter P indicates that the component belongs to a platonic point group, so P₃ represents a tetrahedral point group and P₄ represents an octahedral point group. Capital letter D indicates that the component belongs to a dihedral point group. In a similar manner to Table 1, the final columns give, in respect of each protomer where appropriate, the number M of monomers in the first oligomer assembly and the order(s) N of the set of rotational symmetry axes of the first oligomer assembly which are aligned with the rotational symmetry axis of a second oligomer assembly.

For ease of reference, the protein lattices are divided into classes on the basis of the symmetry of their components, in a similar manner to the division of the protein lattices formed from homologous protomers. In each case, the heterologous protomers may be derived from the protomers of the corresponding class of homologous protomer in Table 1.

For the mixed class and the platonic class, the two types of protomers each comprise:
(a) a first monomer of a homologous oligomer assembly which belongs to the same point group as a respective one of the monomers of the corresponding homologous protomer; and
(b) a second monomer which is a respective one of the two different monomers of the heterologous oligomer assembly which belongs to a cyclic point group.

The order of the cyclic point group to which the heterologous oligomer assembly belongs is the same as the order N of the N-fold fusion between the oligomer assemblies of the protein lattice formed from the corresponding homologous protomer, that is the order of the respective rotational symmetry axis of the first oligomer assembly.

Thus, in the assembled protein lattice, the repeating unit has fundamentally the same arrangement as the repeating unit of the corresponding homologous protomer, except as follows. Instead of the N-fold fusion between the two homologous oligomer assemblies of the homologous protomer, the link between the homologous oligomer assemblies is extended by the insertion of the heterologous oligomer assembly. Therefore, it will be seen that the repeating unit of the heterologous oligomer assembly effectively extends the length of the links of the repeating unit between the first oligomer assemblies which may be considered as nodes in the protein lattice. Thus, the size of the pores within the protein lattice is also increased relative to the use of the corresponding homologous protomers.

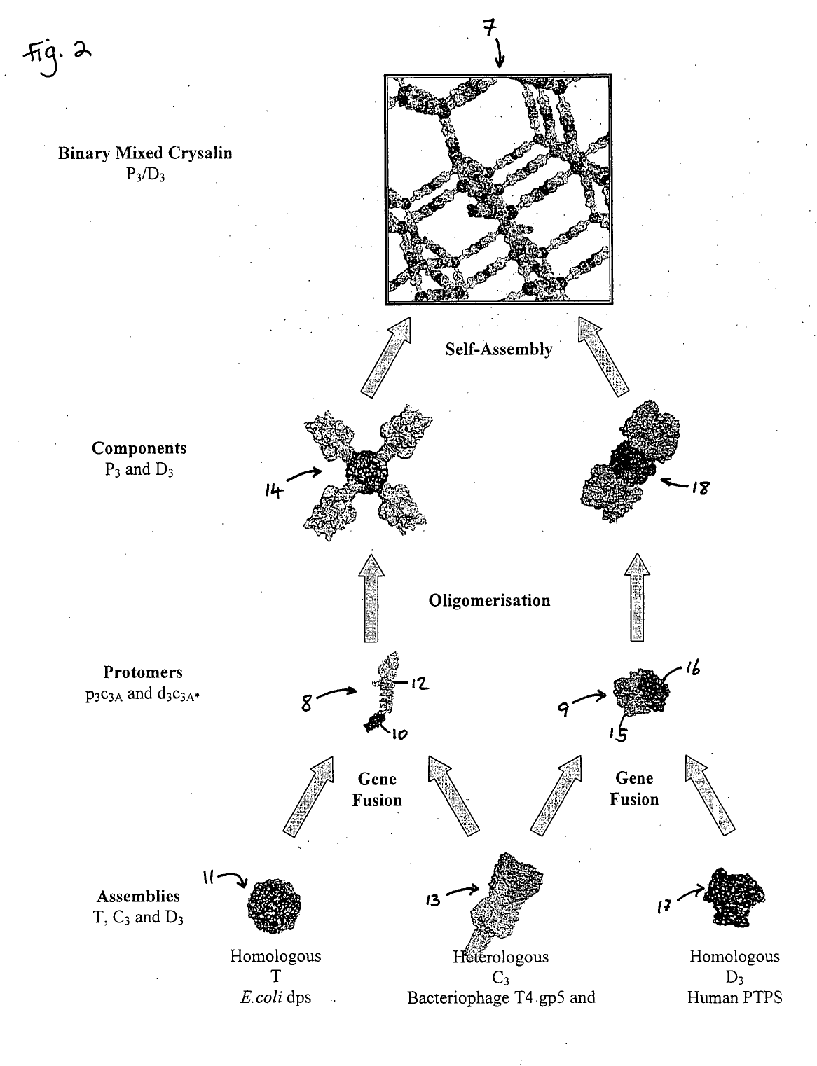

FIG. 2 illustrates a particular example of a protein lattice 7 belonging to the mixed class, in particular having respective protomers 8 and 9 represented by p₃c_{3A} and d₃c_{3A}., respectively. The first protomer 8 comprises a first monomer 10 of a first homologous oligomer assembly 11, namely is E. Coli dps which belongs to a tetrahedral point group. Fused to the first monomer 10 in the first protomer 8 is a second monomer 12 of a further heterologous oligomer assembly 13, namely bacteriophage T4 gp5 and gp27 which belongs to a cyclic point group of order 3. On assembly, the first protomer 8 forms a first component 14 by the first monomers 10 assembling together. The first component 14 has the same symmetry as the first oligomer assembly 11 of the first protomer 8.

The second protomer 9 comprises a monomer 15 which is the other monomer of the second oligomer 13 of the first protomer 8 which is heterologous to the second monomer 12 of the first protomer 8. The second protomer 9 also comprises a monomer 16 which is a monomer of a homologous oligomer assembly 17, namely human PTPS which belongs to a dihedral D₃ point group of order 3. On assembly, the second protomer 9 forms a second component 18 by the homologous monomers 16 assembling together.

When the first and second components 14 and 18 are brought together, they assemble to form the protein lattice 7 by assembly of the heterologous oligomer assembly 13. It is clearly visible from FIG. 2 how the symmetry of the protein lattice 7 is based on the symmetries of the homologous oligomer assemblies 11 and 17. In particular, the rotational symmetry axes of order 3 of both the heterologous oligomer assembly 13 and the homologous oligomer assembly 17 of the second protomer 9 are aligned with the set of rotational symmetry axes of order 3 of the first oligomer assembly 11 of the first protomer 8. It is further clear from FIG. 2 how the heterologous oligomer assemblies 13 effectively extend the length of the links between the first oligomer assemblies 11. In the lattice 7, the repeating unit may be taken, for example, as one of the first components 14 and half of each of the adjacent second components 18. In this case, the unit cell is formed by a number of such repeating units combined together.

The protomers of the dihedral class of the heterologous comprise protomers comprising three monomers which may be derived from a corresponding one of the dihedral class of homologous protomers. In particular, the two types of protomer comprise the corresponding homologous protomer with either one (or both) of the second monomers of the corresponding homologous protomers replaced by respective monomers of a heterologous oligomer assembly belonging to a cyclic point group of the same order as the dihedral point group to which the oligomer assembly of the replaced monomer belongs.

Next there will be described examples to produce a protein layer which repeats in two dimensions.

In these examples, the first oligomer assembly belongs to a dihedral point group of order O, where O equals 2, 3, 4 or 6. Hence the first oligomer assembly has a principal rotational symmetry axis of order O and also O rotational symmetry axes of order 2 which all-extend perpendicular to the principal rotational symmetry axis. In order to develop a layer extending in two dimensions, the second oligomer assembly is chosen to have a rotational symmetry axis of order 2 to align with the O rotational symmetry axes of order 2 of the first oligomer assembly with a 2-fold fusion between the first and second oligomer assemblies. Therefore, in this case, the O rotational symmetry axes of order 2 constitute the set of rotational symmetry axes of the first oligomer assembly, ie N equals O.

In some classes of protein layer, the protomers are homologous with respect to the monomers, ie there is a single type of protomer within the protein lattice. In this case, the first oligomer assembly belongs to a dihedral point group of order O, where O equals 3, 4 or 6. For example, Table 3 represents some simple homologous protomers capable of forming a protein layer.

TABLE 3
Homologous Protomers

| | | | Layer |
|---|---|---|---|
| Protomer | M | N | Symmetry |
| d3d2 | 6 | 2 | P622 |
| d4d2 | 8 | 2 | P422 |
| d6d2 | 12 | 2 | P622 |

In Table 3, each protomer is identified by letters which represent the oligomer assemblies to which the respective monomers of the protomer belong. In particular the letter d represents a dihedral point group and the following number identifies the order of dihedral point group. In the next two columns of the table, there is given the number M of first monomers in the first oligomer assembly and the order N of the set of rotational symmetry axes of the first oligomer assembly which in this case is 2. The final column gives the symmetry of the resulting protein layer. In each of these cases, the second oligomer assembly belongs to a dihedral point group of order 2.

Thus it easy to visualise the protein layers. In particular, the first oligomer assembly may be visualised as a node from which the set of O rotational symmetry axes of order 2 extend outwardly in a common plane, perpendicular to the principal rotational symmetry axis of order O. The second oligomer assemblies may be visualised as linear links extending from the node aligned with respective ones of the set of O rotational symmetry axes of order 2 of the first oligomer assemblies. In this way, it is easy to visualise the formation of the layer with pores in the spaces between the oligomer assemblies. Thus it will be seen that the symmetry of the layer derives from the symmetrical arrangement of the set of O rotational symmetry axes of order 2 of the first oligomer assemblies.

In other classes of protein layer, the protomers are heterologous with respect to the monomers i.e. there are two or more types of protomer in the protein layer. To achieve assembly of any two types of protomer, the two types of protomer include different monomers of the same heterologous oligomer assembly. Thus when the protomers of the different types are allowed to assemble, the heterologous oligomer assemblies assemble, thereby linking the protomers of the two types. However, in contrast to homologous protomers, a single type of protomer cannot by itself assemble into the entire protein layer. The individual monomers of the heterologous oligomer assembly cannot self-assemble into the entire heterologous oligomer assembly in the absence of the other, different monomers of that heterologous assembly. This provides advantages during manufacture of the protein layers, because each type of protomer may be separately produced without assembly of an entire protein layer which might otherwise disrupt the production of the protomer. This allows production in a two-stage process, which will be described in more detail below.

In these examples, the heterologous oligomer assembly is the second oligomer assembly of both types of protomer and belongs to a cyclic point group of order 2. In the simplest types of protein layer, the first oligomer assembly of both types of protomer is a monomer of a homologous oligomer assembly belonging to a dihedral point group. Thus the individual types of protomer may assemble into a respective, discrete component of the unit cell of the repeating pattern, as a result of the monomers of the homologous first oligomer assembly self-assembling. This is an advantage of the heterologous protomers, because assembly of the layer may be avoided until the components are brought together. Otherwise assembly of the layer might hinder the production of the protomers' themselves.

For example, Table 4 represents some simple heterologous protomers capable of forming a protein layer.

TABLE 4
Heterologous Protomers

| | | 1st | | 2nd | | |
|---|---|---|---|---|---|---|
| | | Protomer | | Protomer | | Layer |
| Protomer | Components | M | N | M | N | Symmetry |
| d3c2A + d3c2A* | D3/D3 | 6 | 2 | 6 | 2 | P622 |
| d4c2A + d4c2A* | D4/D4 | 8 | 2 | 8 | 2 | P422 |
| d6c2A + d6c2A* | D6/D6 | 12 | 2 | 12 | 2 | P622 |
| d3c2A + d2c2A* | D3/D2 | 6 | 2 | 4 | 2 | P622 |
| d4c2A + d2c2A* | D4/D2 | 8 | 2 | 4 | 2 | P422 |
| d6c2A + d2c2A* | D6/D2 | 12 | 2 | 4 | 2 | P622 |

In Table 4, the first column identifies the two types of protomer. Each protomer is identified by letters which represent the oligomer assemblies to which the respective monomers of the protomer belong. In particular the letter d represents a dihedral point group and the letter c represents a monomer of a heterologous oligomer assembly belonging to a cyclic point group. The subscript number again represents the order of the point group. The subscript capital letters A and A* are used to identify the two different monomers of the same heterologous assembly.

In Table 4, the second column identifies the point groups to which the components resulting from the assembly of each type of protomer belongs. A similar notation is used as for the monomers of the protomer, except that capital letters are used to indicate that the point group of the component is being referred to. Thus capital letter D indicates that the component belongs to a dihedral point group and the number gives the order of the point group.

In the next four columns of the table, there is given, for each type of protomer, the number M of first monomers in the first oligomer assembly and the order N of the set of rotational symmetry axes of the first oligomer assembly. The final column gives the symmetry of the resulting protein layer.

In all the examples of Table 4, the first oligomer assembly of the first type of protomer belongs to a dihedral point group of order O, where O equals 3, 4 or 6.

In the first three examples of Table 4, the first oligomer assembly of the second type of protomer belongs to a dihedral point group of order L, where L equals O. Thus these three examples have spatially the same arrangement as the three examples of the corresponding homologous protomers in Table 3. In the first three examples of Table 4, the first oligomer assemblies of the two types of protomer may the same oligomer assembly or may be a different oligomer assembly.

In the second three examples of Table 4, the first oligomer assembly of the second type of protomer belongs to a dihedral point group of order L, where L equals 2. These three examples have spatially the same arrangement as the three examples of the corresponding homologous protomers in Table 3, except as follows. Instead of the two dihedral oligomer assemblies of order O being linked by a single cyclic oligomer assembly, the link between the two dihedral oligomer assemblies of order O is extended to be formed by a chain comprising two cyclic oligomer assemblies of order 2 on either side of a dihedral oligomer assembly of order 2. Therefore, it will be seen that the repeating unit of the heterologous oligomer assembly effectively extends the length of the links of the repeating unit between the dihedral oligomer assemblies of order O which may be considered as nodes in the protein layer. Thus, the size of the pores within the protein layer is also increased relative to the use of the corresponding homologous protomers.

Lastly there will be described examples to produce a protein chain which repeats in one dimension. In the case of a protein chain, the first oligomer assembly has rotational symmetry axes which extend in three dimensions.

In these examples, the first oligomer assembly belongs to a dihedral point group of order 2. Hence the first oligomer assembly has two rotational symmetry axes of order 2 extending perpendicular to each other. In order to develop a chain extending in one dimension, the second oligomer assembly is chosen to have a rotational symmetry axis of order 2 to align with one of the rotational symmetry axes of order 2 of the first oligomer assembly with a 2-fold fusion between the first and second oligomer assemblies. Two second oligomer assemblies align with the same rotational symmetry axes of order 2 of the first oligomer assembly but on opposite sides of the first oligomer assembly. Therefore, in this case, one of the rotational symmetry axes of order 2 constitutes the set of rotational symmetry axes of the first oligomer assembly, ie N equals O. The chain develops with the first and second oligomer assemblies alternately arranged.

In some classes of protein layer, the protomers are homologous with respect to the monomers, ie there is a single type of protomer within the protein lattice. In this case, the second oligomer assembly belongs to a dihedral point group of order 2.

In other classes of protein layer, the protomers are heterologous with respect to the monomers i.e. there are two or more types of protomer in the protein layer. This produces the same effects on assembly and manufacture as described above for protein lattices and layers. In these examples, the heterologous oligomer assembly is the second oligomer assembly of both types of protomer and belongs to a cyclic point group of order 2. The first oligomer assemblies of the two types of protomer may the same oligomer assembly or may be a different oligomer assembly.

The above examples of protein structures are believed to represent the simplest form of protomers capable of forming a protein structure and are preferred for that reason. However, it will be appreciated that other protomers formed from monomers of oligomer assemblies having suitable symmetries will be capable of forming a protein structure. For example, other homologous protomers having larger numbers of monomers than listed in Table 1 will be capable of forming a protein lattice. Similarly, other heterologous protomers will be capable of forming a protein lattice. These may include two types of protomer having larger numbers of monomers than in the examples of Table 2, or may include more than two types of protomer.

For each of the monomers, there is a large choice of oligomer assemblies having the required symmetry. The present invention is not limited to particular oligomer assemblies, because in principle any oligomer assembly having a quaternary structure with the requisite symmetry may be used. However, as examples Table 3 lists some possible choices of oligomer assembly for each of the point groups of Tables 1 and 2.

TABLE 3
Example oligomer assemblies

| Point | | | PDB |
|---|---|---|---|
| Group | Source | Name of Oligomer Assembly | Code |
| P₃(T, 32) | E. coli | dps | 1DPS |
| | S. epidermis | EpiD | 1G63 |
| P₄(O, 432) | Human | heavy chain ferritin | 2FHA |
| | E. coli | Dihydrolipoamide succinyl- | 1E20 |
| | | transferase | |
| | A. vinelandii | Dihydrolipoamide acetyl- | 1EAB |
| | | transferase | |
| D₂ | Human | Mn superoxide dismutase | 1AP5 |
| | P. falciparum | lactate dehydrogenase | 1CEQ |
| D₃ | Rat | 6-pyruvoyl tetrahydropterin | 1B66 |
| | | synthase | |
| | E. coli | Amino acid aminotransferase | 1I1L |
| D₄ | E. coli | PurE | 1QCZ |
| | Sipunculid worm | Hemerythrin | 2HMQ |
| D₆ | S. typhimurium | Glutamine Synthetase | 1F1H |
| C_{2A} + C_{2A*} | Human | Casein kinase alpha and | 1JWH |
| | | beta chains | |
| C_{3A} + C_{3A*} | Coliphate T4 | gp5 + gp27 | 1K28 |
| | HIV | N36 + C34 | 1AIK |
| | Pseudomonas | Napthalene 1,2-Dioxygenase | 1NDO |
| | putida | | |
| C_{4A} + C_{4A*} | Erachiopod | Hemerythrin | N/A |

Thus the present invention provides a protein protomer or plural protein protomers capable of assembly into a protein lattice. The monomers of the protomer may be of any length but typically have a length of 5 to 1 000 amino acids, preferably at least 20 amino acids and/or preferably at most 500 amino acids.

The invention also provides polynucleotides which encode the protein protomers of the invention. The polynucleotide will typically also comprise an additional sequence beyond the 5 and/or 3 ends of the coding sequence. The polynucleotide typically has a length of at least three times the length of the encoded protomer. The polynucleotide may be RNA or DNA, including genomic DNA, synthetic DNA or cDNA. The polynucleotide may be single or double stranded.

The polynucleotides may comprise synthetic or modified nucleotides, such as methylphosphonate and phosphorothioate backbones or the addition of acridine or polylysine chains at the 3′ and/or 5′ ends of the molecule.

Such polynucleotides may be produced and used using standard techniques. For example, the comments made in WO-00/68248 about nucleic acids and their uses apply equally to the polynucleotides of the present invention.

The monomers are typically combined to form protomers by fusion of the respective genes at the genetic level (e.g. by removing the stop codon of the 5′ gene and allowing an in-frame read through to the 3′ gene). In this case the recombinant gene is expressed as a single polypeptide. The genes may, alternatively, be fused at a position other than the end terminus so long as the quaternary structure of the oligomer assembly properties remains substantially unaffected. In particular, one gene may be inserted within a structurally tolerant region of a second gene to produce an in-frame fusion.

The invention also provides expression vectors which comprise polynucleotides of the invention and which are capable of expressing a protein protomer of the invention. Such vectors may also comprise appropriate initiators, promoters, enhancers and other elements, such as for example polyadenylation signals which may be necessary, and which are positioned in the correct orientation, in order to allow for protein expression.

Thus the coding sequence in the vector is operably linked to such elements so that they provide for expression of the coding sequence (typically in a cell). The term “operably linked” refers to a juxtaposition wherein the components described are in a relationship permitting them to function in their intended manner.

The vector may be for example, plasmid, virus or phage vector. Typically the vector has an origin of replication. The vector may comprise one or more selectable marker genes, for example an ampicillin resistance gene in the case of a bacterial plasmid or a resistance gene for a fungal vector.

Promoters and other expression regulation signals may be selected to be compatible with the host cell for which expression is designed. For example, yeast promoters include S. cerevisiae GAL4 and ADH promoters, S. pombe nmt1 and adh promoter. Mammalian promoters include the metallothionein promoter which can be induced in response to heavy metals such as cadmium. Viral promoters such as the SV40 large T antigen promoter or adenovirus promoters may also be used.

Mammalian promoters, such as β-actin promoters, may be used. Tissue-specific promoters are especially preferred. Viral promoters may also be used, for example the Moloney murine leukaemia virus long terminal repeat (MMLV LTR), the rous sarcoma virus (RSV) LTR promoter, the SV40 promoter, the human cytomegalovirus (CMV) IE promoter, adenovirus, HSV promoters (such as the HSV IE promoters), or HPV promoters, particularly the HPV upstream regulatory region (URR).

Another method that can be used for the expression of the protein protomers is cell-free expression, for example bacterial, yeast or mammalian.

The invention also includes cells that have been modified to express the protomers of the invention. Such cells include transient, or preferably stable higher eukaryotic cell lines, such as mammalian cells or insect cells, using for example a baculovirus expression system, lower eukaryotic cells, such as yeast or prokaryotic cells such as bacterial cells. Particular examples of cells which may be modified by insertion of vectors encoding for a polypeptide according to the invention include mammalian HEK293T, CHO, HeLa and COS cells. Preferably the cell line selected will be one which is not only stable, but also allows for mature glycosylation of a polypeptide. Expression may be achieved in transformed oocytes.

The protein protomers, polynucleotides, vectors or cells of the invention may be present in a substantially isolated form. They may also be in a substantially purified form, in which case they will generally comprise at least 90%, e.g. at least 95%, 98% or 99%, of the proteins, polynucleotides, cells or dry mass of the preparation.

The protomers may be prepared using the vectors and host cells using standard techniques. For example, the comments made in WO-00/68248 regarding methods of preparing protomers (referred to as “fusion proteins” in WO-00/68248) apply equally to preparation of protomers according to the present invention.

Assembly of the protein lattice from the protomers may be performed simply by placing the protomers under suitable conditions for self-assembly of the monomers of the oligomer assemblies. Typically, this will be performed by placing the protomers in solution, preferably an aqueous solution. Typically, the suitable conditions will correspond to those in which the naturally occurring protein self-assembles in nature. Suitable conditions may be those specifically disclosed in WO-00/68248.

In the case of homologous protomers this results in direct assembly of the protein-lattice.

In the case of heterologous protomers, assembly is preferably performed in plural stages. In a first stage, each type of protomer is separately assembled into a respective discrete component. In a second stage, the discrete components are brought together and assembled into the protein lattice. Where plural heterologous protomers are used, there may be further stages intermediate the first and second stage in which the respective discrete components are brought together and assembled into larger, intermediate components.

A specific protein lattice of the type illustrated in FIG. 1 has been prepared using the following method.

Human ferritin heavy chain (HFH) and the E. coli PurE gene were amplified by PCR from human cDNA and E. coli gDNA respectively. Primers for amplification of the ferritin gene were: 5′-CCT TAG TCG AAT TCA TGA CGA CCG CGT CCA CC-3′ (SEQ ID NO. 3) and 5′-GGG AAA TTA GCC CTC GAG TTA GCT TTC ATT ATC-3′ (SEQ ID NO: 4). Primers for amplification of the PurE gene were: 5′-GTT TTA AGA CCC ATG GCT TCC CGC AAT AAT CCG-3′ (SEQ ID NO. 5) and 5′-CGC AAA CCT GGA TCC TGC CGC ACC TCG CGG-3′ (SEQ ID NO. 6). The PurE gene was cloned into the pET-28b vector (Novagen) between the NcoI and BamHI sites. The HFH gene was cloned into the resulting vector between the EcoRI and XhoI sites to create an in-frame fusion of the two genes under control of the T71ac promoter.

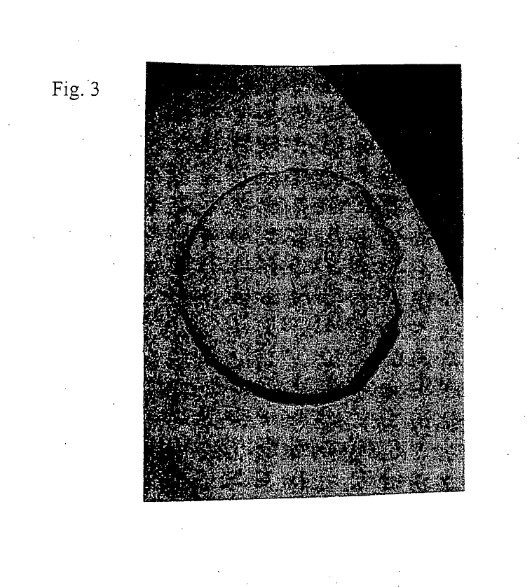

This vector was transformed into E. Coli strain B834(pLysS) for expression. Induction of expression was as follows: a 10 ml overnight culture of the expression strain (in LB broth containing 30 μg/ml Kanamycin) was diluted 1:100 into fresh LB broth containing 30 μg/ml Kanamycin, Cells were grown with shaking at 37° C. to a density corresponding to an OD₆₀₀ of 0.6 and were then induced to express the target protein by the addition of IPTG to a final concentration of 1 mM. The culture was maintained at 37° C. with shaking for a further 3 hours before the cells were harvested by centrifugation (5000 g, 10 min, 4° C.). The cell pellet was resuspended in 20 ml of buffer A (300 mM NaCl, 1 mM EDTA, 50 mM HEPES, pH7.5). Cells were lysed by sonication and the insoluble fraction harvested by centrifugation (25,000 g, 30 min, 4° C.). This fraction was dissolved in 8M urea and centrifuged (25,000 g, 30 min, 4° C.) to remove insoluble particles. The urea solubilised material was concentrated to 16 mg/ml and passed through a 0.22 μm filter. A drop of this material (1 μl) was then directly injected into a larger drop (5 μl) of buffer A. Protein lattice particles were observed within one hour. FIG. 3 is a picture of one of the protein lattice particles having a diameter of approximately 0.6 mm. The elemental composition of the protein lattice has been confirmed using μPIXE techniques.

A specific protein chain has been prepared using the following method.

The protomers consisted of a first monomer being DsRed-Express-StreptagI and a second monomer being Streptavidin. Both monomers assemble into an oligomer assembly belonging to a dihedral point group of order 2. The sequence of the DsRed-Express-Streptag I fusion protein is:

(SEQ ID NO. 1)

MTMITPSLHACRSTLEDPRVPVATMASSEDVIKEFMRFKVRMEGSVNGHE
FEIEGEGEGRPYEGTQTAKLKVTKGGPLPFAWDILSPQFQYGSKVYVKHP
ADIPDYKKLSFPEGFKWERVMNFEDGGVVTVTQDSSLQDGSFIYKVKFIG
VNFPSDGPVMQKKTMGWEASTERLYPRDGVLKGEIHKALKLKDGGHYLVE
FKSIYMAKKPVQLPGYYYVDSKLDITSHEDYTIVEQYERAEGRHHLFLRS
AWRHPQFGG

An oligonucleotide encoding the StreptagI peptide (-A WRHPQFGG) was inserted into the pDsRed-Express vector (Clontech) such that it provided an in frame fusion of the peptide at the C-terminus of the DsRed-Express protein (protein sequence provided below). The protomer was expressed by inoculation of a single colony of BL21 (Star)DE3 E. coli cells containing the plasmid into 500 ml of LB medium containing 75 μg/ml ampicillin in a 2L Erlenmayer flask and shaking of the flask (250 rpm) for 16 hours at 37° C. Cells were lysed by standard procedures, most commonly sonication, in phosphate buffered saline solution.

The soluble protein fraction was isolated by centrifugation (30,000 g, 30 min, 4° C.). Fusion protein was purified using Streptactin MacroPrep affinity chromatography (Stratech Scientific) according to the manufacturers instructions followed by Superose 6 (GE Healthcare) size exclusion chromatography. Protein was eluted in 150 mM NaCl, 50 mM Tr-HCl pH8.0, 1 mM EDTA. Purified protein (1 mg/ml) was mixed with streptavidin (Stratech Scientific) (5 mg/ml in the same buffer solution).

The product was visualised by negative stain electron microscopy using standard procedures and the resulting electron micrograph is shown in FIG. 4. Ferritin molecules were included to provide an internal standard. The protein chains formed can be clearly be seen in FIG. 4.

A specific protein layer has been prepared using the following method.

The protomers consisted of a first monomer being ALAD-StreptagI and a second monomer being Streptavidin. ALAD-StreptagI assembles into an oligomer assembly belonging to a dihedral point group of order 4. Streptavidin assembles into an oligomer assembly belonging to a dihedral point group of order 4. The sequence of the ALAD-Streptag I fusion protein is:

(SEQ ID NO. 2)

MTMGSMTDLIQRPRRLRKSPALRAMFEETTLSLNDLVLPIFVEEEIDDYK
AVEAPGVMRIPEKHLAREIERIANAGIRSVMTFGISHHTDETGSDAWRED
GLVARMSRICKQTVPEMIVMSDTCFCEYTSHGHCGVLCEHGVDNDATLEN
LGKQAVVAAAAGAXFIAPSAAMDGQVQAIRQALDAAGFKDTAIMSYSTKF
ASSFYGPFREAAGSALKGDRKSYQMNPMNRREAIRESLLDEAQGANCLMV
KPAGAYLDIVRELRERTELPIGAYQVSGEYAMIKFAALAGAIDEEKVVLE
SLGSIKRAGADLIFSYFALDLAEKKILRRSAWRHPQFGG

The gene encoding 5-Aminolaevulinic acid dehydratase (ALAD) was amplified from DH5alpha genomic DNA and inserted into the DsRed-Express-streptagI expression vector described above to replace the DsRed-Express gene cassette. An ALAD-streptagI protomer was then prepared by an identical method to that described above for the DsRed-Express-streptagI protein. In this case 0.1 mM IPTG was included in the expression medium.

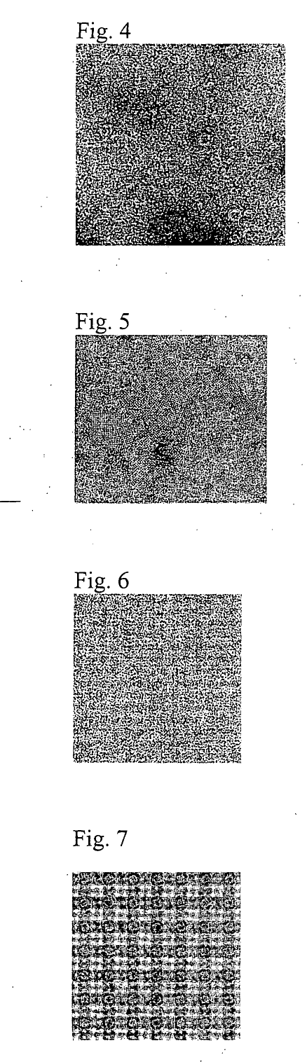

FIG. 4 shows an electron micrographs of resultant product being uranyl acetate stained lattice. Sections of the protein layer are clearly visible. FIG. 5 shows an enlargement of a section of a layer illustrating the repeating pattern of the protein layer, the unit cell size being 13 nm×13 nm.

Image processing of the electron micrographs was performed to enhance the image. In particular the electron micrograph was Fourier transformed, filtered using a space group derived filter and averaging, and then reconstructed. The resultant enhanced image is shown in FIG. 7.

Protein structures in accordance with the present invention have numerous different uses. In general, such uses will take advantage of the regular repeating structure and/or the pores which are present within the structure in the case of a lattice or layer. Lattices or layers in accordance with the present invention may be designed to have pores with dimensions expected to be of the order of nanometres to hundreds of nanometres. Lattices or layers may be designed with an appropriate pore size for a desired use.

The highly defined, unusually sized and finely controlled pore sizes of the protein lattices or layers together with the stability of their structures make them ideal for applications requiring microporous materials with pore sizes in the range just mentioned. As one example, the lattices or layers are expected to be useful as a filter element or molecular sieve for filtration or separation processes. In this use, the pore sizes achievable and the ability to design the size of a pore are particularly advantageous.

In another class of use, molecular entities would be attached to the protein structure. Such attachment may be done using conventional techniques. The molecular entities may be any entities of an appropriate size, typically a macromolecular entity, for example proteins, polynucleotides or non-biological entities. As such, the protein structures are expected to be useful as biological matrices for carrying molecular entities, for example for use in drug delivery, or for crystallizing molecular entities.

Attachment of the molecular entities to the protein structure may be performed by “tagging” either or both of the protein protomers or the molecular entities of interest. In this context, tagging is the covalent addition to either or both of the protein protomers or the target molecular entities, of a structure known as a tag which forms strong interactions with a target structure. Typically, short peptide motifs (e.g. heterodimeric coiled coils such as the “Velcro” acid and base peptides) are used for this purpose. The target structure may be a further tag attached to the other of the protein protomer or target molecular entity, or may be a part of the protein protomer or target molecular entity. In the case of the protein protomer, or a molecular entities which is a protein, this may be achieved by the expression of a genetically modified version of the protein to carry an additional sequence of peptide elements which constitute the tag, for example at one of its termini, or in a loop region.

Alternative methods of adding a tag include covalent modification of a protein after it has been expressed, through techniques such as intein technology.

Thus to attach the molecular entity to the protein structure, the protein protomers may include, at a predetermined position in the protomers, an affinity tag attached to the molecular entity of interest.

Alternatively, the molecular entity of interest may have at a predetermined position in the protomers, an affinity tag attached to a molecular entity.

When a component of the protein structure is known to form strong interactions with a known peptide sequence, that peptide sequence may be used as a tag to be added to the target molecular entity. Where no such tight binding partner is known, suitable tags may be identified by means of screening. The types of screening possible are phage-display techniques, or redundant chemical library approaches to produce a large number of different short (for example 3-50 amino acid) peptides. The tightest binding peptide elements may be identified using standard techniques, for example amplification and sequencing in the case of phage-displayed libraries or by means of peptide sequencing in the case of redundant libraries.

Another approach is to make specific chemical modifications of the lattice in order to provide alternative affinity-based or covalent means of attachment. For example, the site-specific derivitization of accessible sulphydryl groups in the lattice may be used for the incorporation of nitrilo-triacetic acid (NTA) groups which in turn may be used for binding of metal ions and hence histidine rich target proteins.

To attach the molecular entity to the protein structure using an affinity tag on the structure or the molecular entity, the molecular entity may be allowed to diffuse into, and hence become attached to, a pre-formed protein structure, for example by annealing of the bound molecular entity into their lowest energy configurations in the protein structure may be performed using controlled cooling in a liquid nitrogen cryostream. Alternatively, the molecular entities may be mixed with the protomers during formation of the protein structure to assemble with the structure.

Alternatively, the target molecular entity itself may be expressed as a direct genetic fusion to a lattice component.

In another class of uses, proteins having useful properties could be incorporated as one of the protomers.

A use in which an entity is attached to the protein structure is to perform X-ray crystallography of the molecular entities. In this case, the regular structure of the protein lattice allows the molecular entities to be held at a predetermined position relative to a repeating structure, so that they are held in a regular line or array and in a regular orientation. X-ray crystallography is important in biochemical research and rational drug design.

The protein structure having an array of molecular entities supported thereof may be studied using standard x-ray crystallographic techniques. Use of the protein structure as a support in x-ray crystallography is expected to provide numerous and significant advantages over current technology and protocol for X-ray crystallography, including the following:

(1) Significantly lower amounts of molecule will be required (probably of order micrograms rather than milligrams). This will allow determination of some previously intractable targets.

(2) Use of affinity tags will allow structure determination without the typical requirement for a number of purification steps.

(3) There will be no need to crystallize the molecular entity. This is a difficult and occasionally insurmountable step in traditional X-ray structure determination.

(4) There will be no need to obtain crystalline derivatives for each novel crystal structure to obtain the required phase information. Since the majority of scattering matter will be the known protein structure in each case, determination of the structure may be automated and achieved rapidly by a computer user with little or no crystallographic expertise.

(5) The complexes of a protein with chemicals (substrates/drugs) and with other proteins can be examined without requiring entirely new crystallization conditions.

(6) The process is expected to be extremely rapid and universally applicable, which will provide enormous savings in time and costs.

Another use in which an entity is attached to the protein structure is to perform electron microscopy of the molecular entities. This may be performed to determine the structure of the entities. In this case, particular advantage is obtained by the use of a protein layer or chain. The electron microscopy may be performed as follows.

For optimal resolution in the structure of the molecular entity, it is preferable for the molecular entities to be aligned with identical orientations with respect to every axis. Two methods of molecular alignment may be implemented either independently or in combination. Firstly, an electric field with a vector parallel to the principal symmetry axis of the “first” protein structure component may be employed in order to align the molecular entities by virtue of their intrinsic or induced dipoles. Secondly, it may be possible to take advantage of polar and/or hydrophobic interactions between molecular entities and the protein structure through a process of thermal annealing during which the target molecules are slowly cooled to identical minimum energy conformations.

Regardless of the orientation procedure adopted (if any), the sample is prepared for viewing by means of an electron microscope by standard procedures (using either cryo-cooling or negative staining with a heavy-atom salt). Sample imaging is also conducted using standard protocols. Images are collected at a series of defocus steps and also employing the tilt-stage of the microscope to image the lattice through a range of angles. Where orientation of the target molecules has been successful, a series of electron diffraction images may also be usefully collected.

Recovery of 3D structural information from images of protein structures and attached molecular entities is achieved using a combination of established protocols for the analysis of electron micrographs of molecular species. For 1D periodic arrays, the main approach is “helical reconstruction”, while for 2D periodic arrays, the most widely applied technique is termed “2D crystallography. For isolated molecules, the approach taken is termed “single particle image reconstruction”.

Single particle image reconstruction tools can also theoretically be applied to image reconstruction of 1D and 2D periodic arrays, and where this provides improved image reconstruction, that approach is also taken to image protein structures and attached molecular entities. Hybrid methods, whereby some computational techniques of 2D crystallography are combined with computational techniques of single particle image analysis, are also used where this is suitable.

If the molecular entities do not adopt the crystalline order and symmetry of their host protein structure, it is possible to apply single particle methods to achieve their image reconstruction. In this case, one or more recorded images of a protein structure and attached molecular entities is analysed initially to image the underlying protein structure. The contribution of the protein structure to the recorded images is then subtracted to generate images that correspond to the molecular entities in isolation. These are then analysed using single particle image reconstruction techniques. This process is expedited by the fact that the protein structure will be found at readily predicted positions on the image, as a consequence of their binding to known locations on the protein structure, the location and orientation of which is readily identified.

For use in catalysing biotransformations, enzymes may be attached to the protein structure, or incorporated in the protein structure.

For use in data storage, it may be possible to attach a protein which is optically or electronically active. One example is Bacteriorhodopsin, but many other proteins can be used in this capacity. In this case, the protein structure holds the attached protein in a highly ordered array, thereby allowing the array to be addressed. The protein structure might overcome the size limitations of existing matrices for holding proteins for use in data storage.

For use in a display, it may be possible to attach a protein which is photoactive or fluorescent. In this case, the protein structure holds the attached protein in a highly ordered array, thereby allowing the array to be addressed for displaying an image.

For use in charge separation, a protein which is capable of carrying out a charge separation process may be attached to the protein structure, or incorporated in the protein structure. Then the protein may be induced to carry out the separation, for example biochemically by a “fuel” such as ATP or optically in the case of a photoactive centre such as chlorophyll or a photoactive protein such as rhodopsin. A variety of charge separation processes might be performed in this way, for example ion pumping or development of a photo-voltaic charge.

For use as a nanowire, a protein which is capable of electrical conduction may be attached to the protein structure, or incorporated in the protein structure. Using an anisotropic protein structure, it might be able to provide the capability of carrying current in a particular direction.

For use as a motor, proteins which are capable of induced expansion/contraction may be incorporated into the protein structure.

The protein lattices may be used as a mould. For example, silicon could be diffused or otherwise impregnated into the pores of the protein lattice, thus either partially or completely filling the lattice interstices. The protein material comprising the original lattice may, if required, then be removed, for example, through the use of a hydrolysing solution.

1. A protein structure which repeats regularly in one, two or three dimensions,
the protein structure comprising protein protomers which each comprise at least two monomers genetically fused together, the monomers each being monomers of a respective oligomer assembly, the protomers comprising:
a first monomer which is a monomer of a first oligomer assembly having rotational symmetry axes extending in at least two dimensions, including a set of rotational symmetry axes of order N, where N equals 2, 3, 4 or 6; and
a second monomer genetically fused to said first monomer which second monomer is a monomer of a second oligomer assembly having a rotational symmetry axis of the same order N as said set of rotational symmetry axes of said first oligomer assembly,
the first monomers of the protomers are assembled into said first oligomer assemblies and the second monomers of the protomers are assembled into said second oligomer assemblies, said rotational symmetry axis of said second oligomer assemblies of order N being aligned with one of said set of rotational symmetry axes of order N of one of said first oligomer assemblies with N protomers being arranged symmetrically therearound, the arrangements of the rotational symmetry axes of the first oligomer assembly and the second oligomer assembly causing the protein structure to repeat regularly in one, two or three dimensions.

2. A protein structure according to claim 1, being a protein lattice which repeats regularly in three dimensions.

3. A protein structure according to claim 2, wherein the protomers are homologous with respect to the monomers.

4. A protein structure according to claim 3, wherein said first oligomer assembly belongs to either a tetrahedral point group or an octahedral point group.

5. A protein structure according to claim 4, wherein said second oligomer assembly belongs to a dihedral point group.

6. A protein structure according to claim 4, wherein said second oligomer assembly belongs to either a tetrahedral point group or an octahedral point group.

7. A protein structure according to claim 3, wherein said first oligomer assembly belongs to a dihedral point group of order O, where O equals 3, 4 or 6, said set of rotational symmetry axes being constituted by a set of one rotational symmetry axis of order O and the first oligomer assembly having a further set of O rotational symmetry axes of order 2,
said protomers comprising:
said first monomer;
said second monomer genetically fused to one terminus of said first monomer; and
a third monomer genetically fused to the other terminus of said first monomer which third monomer is a monomer of a respective third oligomer assembly having a rotational symmetry axis of the same order 2 as said further set of O of rotational symmetry axes of said first oligomer assembly, the third monomers of the protomers being assembled into said third oligomer assemblies, said rotational symmetry axis of said third oligomer assemblies of order 2 being aligned with one of said set of O rotational symmetry axes of order 2 of one of said first oligomer assemblies with 2 protomers being arranged symmetrically therearound.

8. A protein structure according to claim 7, wherein said second monomer is a monomer of an oligomer assembly which belongs to a dihedral point group of order O.

9. A protein structure according to claim 7, wherein said third monomer is a monomer of an oligomer assembly which belongs to a dihedral point group of order 2.

10. A protein structure according to claim 2, wherein the protomers are heterologous with respect to the monomers.

11. A protein structure according to claim 10, wherein the protein structure includes protein protomers of two types, each type comprising:
a first monomer which is a monomer of a first oligomer assembly having rotational symmetry axes extending in at least two dimensions, including a set of rotational symmetry axes of order N, where N equals 2, 3, 4 or 6; and
a second monomer genetically fused to said first monomer which second monomer is a monomer of a second oligomer assembly having a rotational symmetry axis of the same order N as said set of rotational symmetry axes of said first oligomer assembly,
wherein the second monomers of each type of protomer are different monomers of the same heterologous oligomer assembly.

12. A protein structure according to claim 11, wherein said heterologous oligomer assembly belonging to a cyclic point group.

13. A protein structure according to claim 12, wherein the first oligomer assembly of the first type of protomer belongs to either a tetrahedral point group or an octahedral point group.

14. A protein structure according to claim 13, wherein the first oligomer assembly of the second type of protomer belongs to a dihedral point group of the same order as said heterologous oligomer assembly.

15. A protein structure according to claim 13, wherein the first oligomer assembly of the second type of protomer belongs to either a tetrahedral point group or an octahedral point group.

16. A protein structure according to claim 1, being a protein layer which repeats regularly in two dimensions.

17. A protein structure according to claim 16, wherein the protomers are homologous with respect to the monomers.

18. A protein structure according to claim 17, wherein said first oligomer assembly belongs to a dihedral point group of order O, where O equals 3, 4 or 6, said set of rotational symmetry axes being constituted by a set of O rotational symmetry axes of order 2, and said second oligomer assembly belongs to a dihedral point group of order 2.

19. A protein structure according to claim 16, wherein the protomers are heterologous with respect to the monomers.

20. A protein structure according to claim 19, wherein the protein structure includes protein protomers of two types, each type comprising:
a first monomer which is a monomer of a first oligomer assembly belonging to a dihedral point group, the first oligomer assembly of the first type of protomer belonging to a dihedral point group of order O, where O equals 3, 4, or 6, and the first oligomer assembly of the second type of protomer belonging to a dihedral point group of order L, where L equals 2 or O; and
a second monomer genetically fused to said first monomer which second monomer is a monomer of a second oligomer assembly, the second monomers of the first and second types of protomer being different monomers of the same heterologous oligomer assembly belonging to a cyclic point group of order O.

21. A protein structure according to claim 1, being a protein chain which repeats regularly in one dimension.

22. A protein structure according to claim 21, wherein the protomers are homologous with respect to the monomers.

23. A protein structure according to claim 22, wherein said first oligomer assembly belongs to a dihedral point group of order O, where O equals 2, 3, 4 or 6, said set of rotational symmetry axes being constituted by a set of one rotational symmetry ax1s of order O, and said second oligomer assembly belongs to a dihedral point group of order O.

24. A protein structure according to claim 21, wherein the protomers are heterologous with respect to the monomers.

25. A protein structure according to claim 19, wherein the protein structure includes protein protomers of two types, each type comprising:
a first monomer which is a monomer of a first oligomer assembly belonging to a dihedral point group, the first oligomer assembly of the first type of protomer belonging to a dihedral point group of order O, where O equals 2, 3, 4, or 6, and the first oligomer assembly of the second type of protomer belonging to a dihedral point group of order O; and
a second monomer genetically fused to said first monomer which second monomer is a monomer of a second oligomer assembly, the second monomers of the first and second types of protomer being different monomers of the same heterologous oligomer assembly belonging to a cyclic point group of order O.

26. A protein structure according to claim 1, wherein each of said monomers of said respective oligomer assemblies either is a naturally occurring protein or is based on a naturally occurring protein with peptide elements being absent from, substituted in, or added to the naturally occurring protein without substantially affecting assembly of monomers of said respective oligomer assembly.

27. A protein structure according to claim 1, wherein, in said protomers, said monomers are genetically fused via a linking group.

28. A protein structure according to claim 27, wherein the linking group is oriented relative to the first and second monomers in the protomer in its normal form prior to assembly to reduce any difference in the assembled structure in either or both of the position and orientation of (a) the termini of said first monomers in their arrangement in said first oligomer assembly in its natural form symmetrically around said one of said set of rotational symmetry axes of order N of said first oligomer assembly, and (b) the termini of said second monomers in their arrangement in said second oligomer assembly in its natural form symmetrically around said rotational symmetry axis of order N of said second oligomer assembly.

29. A protein structure according to claim 1 having an array of molecular entities attached thereto.

30. A protein structure according to claim 29, wherein the protomers have, at a predetermined position in the protomers, an affinity tag, the molecular entities being attached to respective affinity tags.

31. A protein structure according to claim 29, wherein the molecular entities have a peptide affinity tag attached to one of the protomers in the protein structure.

32. A method of performing x-ray crystallography or electron microscopy, comprising:
supporting an array of molecular entities on a protein structure according to claim 1; and
performing x-ray crystallography or electron microscopy on the protein structure having the molecular entities supported thereon to derive image data.

33. A protein protomer comprising at least two monomers genetically fused together, the monomers each being monomers of a respective oligomer assembly into which the monomers are capable of self-assembly to assemble a protein structure which repeats regularly in one, two or three dimensions, wherein said protomer comprises:
a first monomer which is a monomer of a first oligomer assembly having rotational symmetry axes extending in at least two dimensions, including a set of rotational symmetry axes of order N, where N equals 2, 3, 4 or 6; and
a second monomer genetically fused to said first monomer which second monomer is a monomer of a second oligomer assembly having a rotational symmetry axis of the same order N as said set of rotational symmetry axes of said first oligomer assembly.

34. A polynucleotide encoding a protein protomer according to claim 33.

35. A vector capable of expressing a protomer according to claim 33.

36. A host cell comprising a vector according to claim 35.

37. Plural different protein protomers, each comprising at least two monomers genetically fused together, the monomers each being monomers of a respective oligomer assembly into which the monomers are capable of self-assembly to assemble from the plural different protein protomers a protein structure which repeats regularly in one, two or three dimensions, wherein each protomer comprises:
a first monomer which is a monomer of a first oligomer assembly having rotational symmetry axes extending in at least two dimensions, including a set of rotational symmetry axes of order N, where N equals 2, 3, 4 or 6; and
a second monomer genetically fused to said first monomer which second monomer is a monomer of a second oligomer assembly having a rotational symmetry axis of the same order N as said set of rotational symmetry axes of said first oligomer assembly.

38. A polynucleotide encoding one of the plural different protein protomers according to claim 37.

39. A vector capable of expressing a protomer according to claim 38.

40. A host cell comprising a vector according to claim 39.
